(19) United States
(12) Patent Application Publication
(10) Pub. No.: US 2026/0330493 A1
(43) Pub. Date: Oct. 22, 2026

(54) CO-PAY MESSAGE DISTRIBUTION SYSTEMS AND METHODS

(71) Applicant: HARSHIT JAIN, Short Hills, NJ (US)

(72) Inventor: HARSHIT JAIN, Short Hills, NJ (US)

(21) Appl. No.: 19/184,031

(22) Filed: Apr. 21, 2025

(51) Int. Cl. G06Q 30/0207; G06Q 40/08; G16H 20/10; G16H 70/40

(52) U.S. Cl. CPC G06Q 30/0213; G06Q 40/08; G16H 20/10; G16H 70/40

(57) ABSTRACT

A co-pay discount delivery system is disclosed that integrates with an electronic prescription interface within an electronic health record (EHR) system or point-of-care (POC) platform. When a healthcare professional enters a prescription including a National Drug Code (NDC), the system automatically triggers a request to a remote discount platform. The discount platform retrieves available co-pay offers from one or more publisher sources and returns a branded or generic discount, if available. The system then enables delivery of the discount to the patient and pharmacy via multiple channels, including pharmacy notes, patient portals, text messages, email, and printed output. The discount may be embedded into a pharmacy instruction to allow application of the benefit at the point of sale. The system further tracks discount utilization to support reporting and analytics. These features collectively reduce patient out-of-pocket prescription costs and improve access to affordability programs in real time during clinical care.

### BACKGROUND

According to a recent study in the United States, patient out-of-pocket healthcare expenses rose by $1 billion, reaching a record high of $77 billion. In parallel, spending on co-pay electronic discount messages tied to affordability programs also peaked at $14 billion. Despite these rising expenditures, patient adoption remained low—only 16% of available co-pay electronic discounts were actually used. Consequently, billions of dollars in potential patient savings went unclaimed, representing a significant loss in available financial support for medical treatment.

Furthermore, recent trends indicate that patients are spending increasingly large amounts to obtain necessary medical care, while expenditures on co-pay electronic discounts continue to rise at an exponential rate. Despite the growing availability of these affordability programs, many patients are unable to benefit from them. A significant number of individuals discontinue treatment due to the high cost of prescribed medications. Additionally, a widespread lack of trust in insurance providers further deters patients from engaging with co-pay programs. As a result, many patients fail to utilize the electronic discounts intended to reduce their financial burden under various affordability initiatives.

### BRIEF DESCRIPTION OF THE DRAWINGS

FIG. 1 shows a block diagram of an overview of a co-pay message distribution of one embodiment.

FIG. 2 shows for illustrative purposes only an example of an EHR triggering a electronic discount request of one embodiment.

FIG. 3 shows a block diagram of an overview of the Co-pay Messaging Distribution Channels of one embodiment.

FIG. 4A shows a block diagram of an overview of a electronic discount request platform of one embodiment.

FIG. 4B shows a block diagram of an overview of pharmacy notes and the affordability message of one embodiment.

FIG. 5 shows a block diagram of an overview of a prescription confirmation of one embodiment.

FIG. 6 shows a block diagram of an overview of the distribution of electronic discounts to the patient portal of one embodiment.

FIG. 7 shows a block diagram of an overview of electronic discount text message distribution of one embodiment.

FIG. 8 shows a block diagram of an overview of a distribution of electronic discounts in the form of a printout of one embodiment.

FIG. 9 shows a block diagram of an overview of a electronic discount email distribution of one embodiment.

FIG. 10 shows a block diagram of an overview of a electronic discount request call of one embodiment.

FIG. 11 shows a block diagram of an overview of the publisher platform of one embodiment.

FIG. 12 shows a block diagram of an overview of a electronic discount response of one embodiment.

FIG. 13 shows a block diagram of an overview of the benefits of co-pay messaging of one embodiment.

### DETAILED DESCRIPTION OF THE INVENTION

In the following description, reference is made to the accompanying drawings, which form a part hereof, and which are shown by way of illustration a specific example in which the invention may be practiced. It is to be understood that other embodiments may be utilized, and structural changes may be made without departing from the scope of the present invention.

It should be noted that the descriptions that follow, for example, in terms of a co-pay message distribution are described for illustrative purposes and the underlying system can apply to any number and multiple types of prescription medications. In one embodiment of the present invention, the co-pay message distribution can be configured using vendor electronic discounts, discount cards, and other forms of product discount price savings. The co-pay message distribution can be configured to include branded drugs and can be configured to include generic drugs using the present invention.

The invention is a co-pay discount delivery system comprising a processor operatively coupled to the electronic prescription page of a healthcare professional's (HCP) Electronic Health Record (EHR) system or Point-of-Care (POC) application, where the EHR or POC represents the clinical software platform accessed by the HCP during patient encounters. The system is configured to automatically trigger an electronic discount request when the HCP enters a prescription that includes a National Drug Code (NDC) identifying the medication. A discount request processor, coupled to the initial processor and further linked to at least one publisher platform, is configured to integrate with the EHR or POC application. This processor is further configured to retrieve applicable co-pay message electronic discounts from the publisher platform, thereby enabling HCPs to access and distribute relevant affordability offers in real time.

A co-pay message distribution system is operatively integrated with the Electronic Health Record (EHR) system or Point-of-Care (POC) platform and is configured to provide healthcare professionals (HCPs) with access to patient-specific cost-saving and affordability program electronic discounts during active caregiving sessions. A server, coupled to the co-pay message distribution module, is further configured to manage and distribute both branded and generic medication discounts and to offer multiple delivery channels through which the HCP may transmit these co-pay messages to the patient and/or the pharmacy.

A database is coupled to a tracking module integrated with at least one publisher platform, the module configured to monitor and log the frequency with which patients redeem electronic discounts for electronically prescribed medications. This data, along with related revenue and performance metrics, is stored in the database for analysis and reporting. Additionally, a processor interfaced with a publisher-linked pharmacy system is configured to identify and select a pharmacy location convenient to the patient for fulfillment of the electronic prescription. This processor facilitates application of the associated co-pay discount to the patient's bill at the selected pharmacy before dispensing the medication. An analytics engine, coupled to the co-pay message distribution module, is further configured to evaluate the extent of out-of-pocket cost reduction achieved through the applied discount. The engine transmits this affordability data to the HCP to support treatment decisions and encourage ongoing patient adherence to the prescribed therapeutic regimen.

As used herein, the term “electronic discount” in singular and plural is used interchangeably with the term “coupon” in singular and plural without any change in meaning. As used herein, the term “eRx” is used interchangeably with the term “electronic prescription” without any change in meaning.

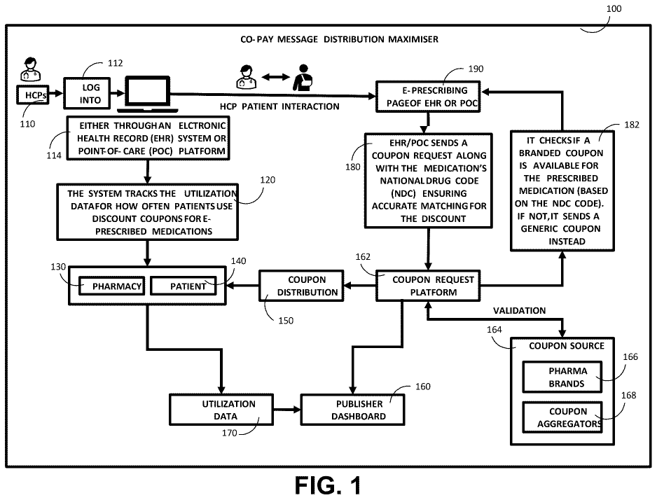

FIG. 1 illustrates a block diagram providing an overview of one embodiment of the co-pay message distribution system 100. In this embodiment, the system is configured to enable a healthcare professional (HCP) to access and deliver co-pay electronic discounts that reduce prescription costs for patients. HCPs 110 interact with the system by logging into 112 a patient care interface, which may be implemented through an Electronic Health Record (EHR) system or a Point-of-Care (POC) platform 114. Once engaged, the system enables real-time access to available co-pay offers and tracks utilization data 170 reflecting how frequently patients redeem the electronic discounts for electronically prescribed medications 120. The system is further configured to facilitate distribution of the co-pay messages to both the pharmacy 130 and the patient 140, ensuring that the discounts are readily accessible through various supported delivery channels, thereby streamlining the prescription fulfillment and affordability process.

The system further comprises a tracking and reporting interface, referred to as the publisher dashboard 160, which is configured to display real-time electronic discount utilization data and associated revenue-related metrics. This dashboard is operatively linked to the electronic discount request platform 162. The system also includes a source validation module 164 that verifies the origin of the electronic discounts, including discounts supplied by pharmaceutical manufacturers 166 and aggregators of co-pay offers for both branded and generic medications 168. The electronic discount request platform 162 is integrated directly into the HCP's 110 Electronic Health Record (EHR) or Point-of-Care (POC) system. Upon initiation of a treatment or caregiving session, the system automatically triggers a discount query when the HCP enters a prescription that includes a National Drug Code (NDC). This query is transmitted to the request platform to identify and retrieve a matching electronic discount 180, if one is available, for subsequent delivery through the configured distribution channels.

When the EHR or POC system transmits an electronic discount request from its e-prescribing interface to the electronic discount request platform 162, the platform evaluates the request by referencing the National Drug Code (NDC) associated with the prescribed medication. The platform first determines whether a branded co-pay electronic discount is available for the specified NDC. If no branded discount is available, the system automatically retrieves and returns a generic co-pay electronic discount 182. The selected discount is then transmitted back to the e-prescribing interface 190 of the initiating EHR or POC system for presentation to the HCP. These electronic discounts serve to reduce the patient's out-of-pocket medication costs, thereby improving affordability and supporting continuity of care. The entire process is initiated when the HCP logs into the EHR or POC system to manage the patient encounter and prescribe treatment.

During a caregiving session, the healthcare professional (HCP) engages in a clinical consultation with the patient. As part of this interaction, the co-pay discount delivery system enables real-time identification and transmission of applicable affordability program benefits directly to the patient. By delivering co-pay discounts at the point of care—while the HCP is actively prescribing—the system helps mitigate the financial burden of high prescription costs. This real-time benefit delivery increases the likelihood that patients will receive and utilize available discounts, improving access to prescribed therapies. As a result, patients are more likely to adhere to their treatment regimens, leading to improved clinical outcomes and reinforcing the trust and continuity in the HCP-patient relationship.

Additionally, the ability to deliver co-pay benefits during the caregiving session directly contributes to improved medication adherence by lowering the patient's out-of-pocket expenses at the point of prescribing. To address and optimize the historically low utilization of co-pay messages associated with various affordability programs, the co-pay message distribution system provides HCPs with access to a wide array of cost-saving opportunities and incentive-based electronic discounts. These benefits can be seamlessly delivered to patients in real time during HCP-patient interactions, enabling healthcare professionals to proactively support treatment adherence by minimizing financial barriers at the point of care.

The co-pay message distribution system enables its supply and publisher partners to generate contextually relevant co-pay messages based on the specific prescription and clinical context. These messages may include both branded and generic electronic discount offers. Once generated, the co-pay messages are distributed through integrated Electronic Health Record (EHR) systems or Point-of-Care (POC) platforms and made available to patients during their clinical encounters. Patients can then utilize these electronic discounts at participating network pharmacies to access affordability program benefits and reduce their out-of-pocket prescription costs.

The co-pay messages, in the form of electronic discounts, are generated based on the specific prescription issued by the healthcare professional (HCP) through their integrated Electronic Health Record (EHR) system or Point-of-Care (POC) platform. These messages are dynamically triggered and displayed on the electronic prescription (eRx) page in real time, corresponding to the medication selected by the HCP. If an eligible electronic discount is available for the prescribed drug at the time of prescribing, the system automatically identifies and presents the discount for distribution to the patient through the configured delivery channels. The EHR, POC, or integrated Health System connected to the electronic discount request platform is configured to support multiple distribution channels for delivering co-pay program electronic discounts. These supported channels include: (1) Pharmacy Notes, (2) Patient Portal, (3) Text Messages, (4) Email, and (5) Print. The selection and configuration of these delivery pathways may be predefined by the publisher or customized based on system integration parameters.

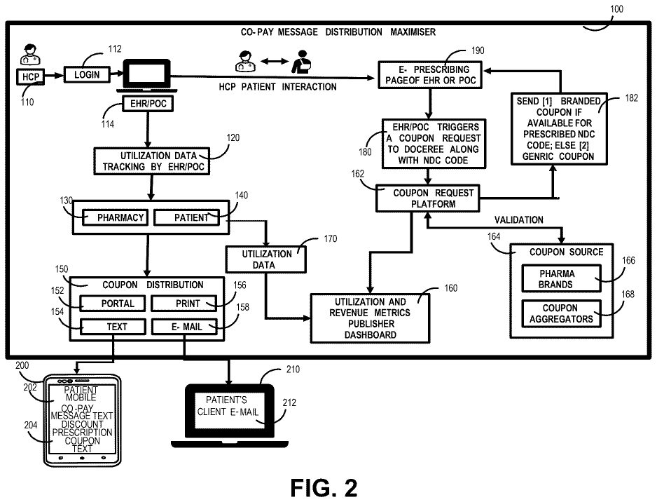

FIG. 2 illustrates, for example purposes only, an embodiment in which an Electronic Health Record (EHR) or Point-of-Care (POC) system initiates an electronic discount request. In this embodiment, the co-pay message distribution system 100 is activated when the healthcare professional (HCP) 110 logs into 112 the EHR or POC platform 114. As part of this interaction, the system begins tracking utilization data 120 associated with discount usage. Once a prescription is issued, the electronic discount may be delivered to the patient 140 for use at a pharmacy 130 through one or more distribution channels 150, including a patient portal 152, text message 154, printed output 156, or email 158. Utilization of the electronic discount is recorded in the system's tracking module 170 and reported on the publisher dashboard 160, which is integrated with the electronic discount request platform 162. The platform also includes a validation module 164 configured to verify the origin of the discount, whether sourced from pharmaceutical manufacturers 166 or co-pay discount aggregators 168, for both branded and generic medications.

Utilization data 170 is generated when the EHR or POC system initiates an electronic discount request from the electronic prescription (eRx) page 190. This request includes the medication's National Drug Code (NDC), which is used by the system to identify and match the prescribed drug with an available co-pay discount 180. The electronic discount request platform then queries one or more data sources to determine if a branded electronic discount is available for the given NDC. If a branded discount is found, it is returned to the system; otherwise, a generic electronic discount 182 is selected and provided to the HCP 110. The selected discount is then delivered to the patient through one or more distribution channels configured by the publisher. These may include sending a co-pay text message 204 to the patient's mobile device 200, delivering an email 158 to the patient's computer 210, posting the discount to the patient portal 152, or printing the discount at the HCP's location 156 for in-person distribution.

The electronic discount distribution process is initiated when the healthcare professional (HCP) accesses the electronic prescription (eRx) page within the EHR or POC system and enters a prescription for the patient. Entry of a medication—typically identified by a standardized code such as a National Drug Code (NDC) or RxNorm identifier—automatically triggers the co-pay message distribution workflow. In response, the EHR or POC system generates and transmits an electronic discount request to the electronic discount request platform. This request includes the NDC and other relevant prescribing details necessary to identify and retrieve a corresponding co-pay discount for the prescribed medication.

The electronic discount request platform is integrated with a network of pharmaceutical manufacturers and electronic discount aggregators, which supply the platform with up-to-date co-pay discount availability and utilization data. When the platform receives an electronic discount request from the EHR or POC system containing a National Drug Code (NDC), it queries these integrated sources to determine whether a corresponding electronic discount is available. If a matching discount—typically a branded co-pay offer—is found, the platform retrieves the relevant discount data and returns it to the originating EHR or POC system. The discount is then prepared for delivery through one or more distribution channels as preconfigured by the publisher partner.

If the electronic discount request platform is unable to locate a branded co-pay discount corresponding to the submitted National Drug Code (NDC), it defaults to retrieving a generic electronic discount. This generic discount is then transmitted back to the originating EHR or POC system and is distributed to the patient through the delivery channels previously configured by the publisher partner. Once the discount is delivered, the electronic discount request platform begins capturing and aggregating relevant utilization data and associated revenue metrics. These metrics are continuously updated and made available in real time for monitoring and analysis via the Publisher Dashboard. The EHR or POC system distributes the co-pay electronic discount received from the electronic discount request platform through preconfigured distribution channels, which in one embodiment include: Pharmacy Notes, Patient Portal, Text Messages, Email, and Print.

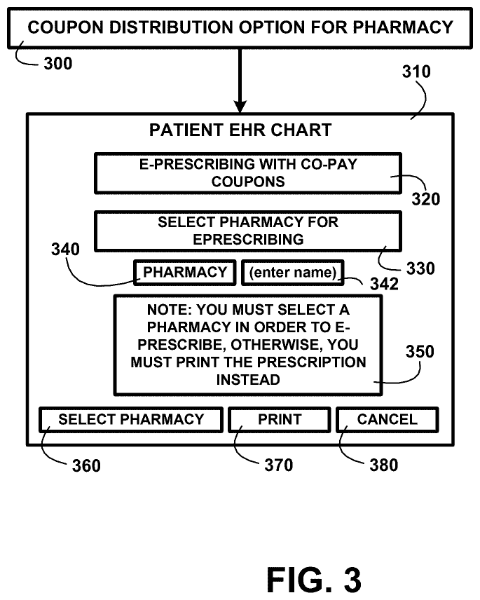

FIG. 3 presents a block diagram illustrating an overview of the co-pay messaging distribution channels in accordance with one embodiment. In this embodiment, an electronic discount distribution option is depicted for pharmacy 300, with the corresponding discount information being linked to the patient's Electronic Health Record (EHR) chart 310. As part of the e-prescribing workflow 320, the healthcare professional (HCP) 110 is prompted to select a pharmacy for electronic prescription routing 330. The HCP may manually enter a pharmacy name 340 or choose from predefined options 360. A system-generated reminder 342 notifies the HCP that a pharmacy selection is required to proceed with e-prescribing; otherwise, the prescription must be printed 350. The HCP is presented with selectable options, including proceeding with pharmacy selection 360, opting to print the prescription 370, or canceling the process entirely 380.

Within the affordability messaging framework, it is important to note that there are three mandatory distribution channels through which electronic discount messages must be delivered by publisher partners: (1) Pharmacy Notes, (2) Patient Portal, and (3) Text Messages. In addition to these required channels, publisher partners may also configure optional communication methods for delivering co-pay discount benefits to patients. These optional channels include: (4) Print and (5) Email. The selection and configuration of these distribution pathways are determined during system integration based on publisher preferences and workflow requirements.

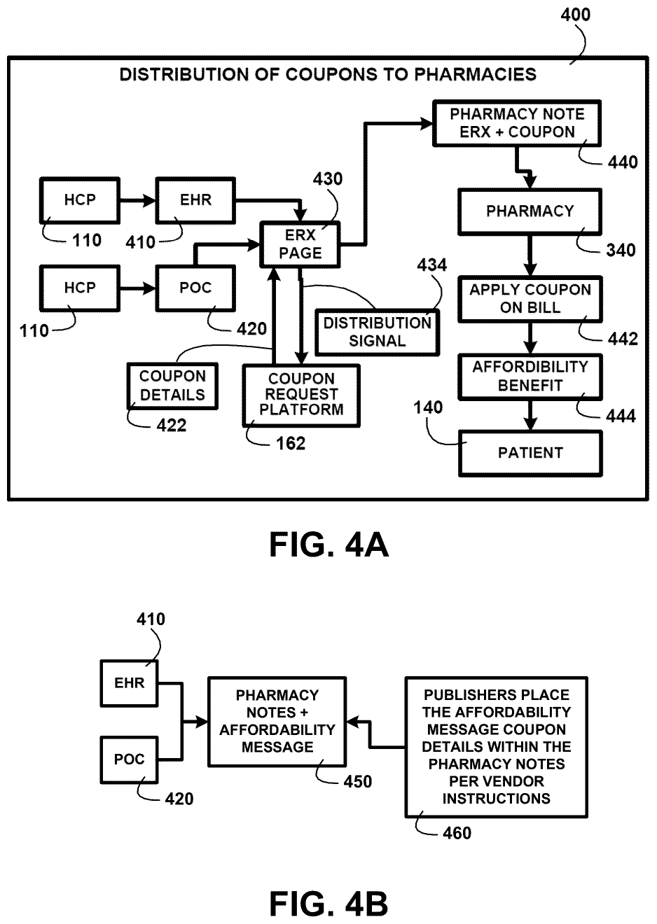

FIG. 4A illustrates a block diagram of one embodiment of the electronic discount request platform. In this embodiment, the platform facilitates the distribution of electronic discounts to pharmacies 400. When the healthcare professional (HCP) 110 accesses the eRx page 430 via either the EHR system 410 or Point-of-Care (POC) system 420, the eRx page generates and transmits a distribution signal 434 to the electronic discount request platform 162. Upon receiving the signal, the platform processes the request and returns the corresponding electronic discount details 422 to the eRx interface. These discount details are then integrated into a pharmacy note that combines the prescription and the electronic discount 440. The completed pharmacy note is transmitted to the designated pharmacy 340, accompanied by instructions to apply the electronic discount to the patient's bill 442 and deliver the associated affordability benefit 444 to the patient 140.

FIG. 4B illustrates a block diagram of one embodiment showing how pharmacy notes are generated along with the corresponding affordability message. In this embodiment, the Electronic Health Record (EHR) system 410 or Point-of-Care (POC) system 420 transmits a combined output containing both the pharmacy note and the co-pay affordability message 450. Publishers are responsible for embedding the electronic discount details into the pharmacy note in accordance with vendor-specific formatting and integration instructions 460, which may be based on financial parameters or business logic related to the discount offering. To facilitate distribution of electronic discounts to pharmacies, the e-prescription (eRx) page—within an EHR or POC system integrated with the electronic discount request platform—must include the co-pay discount information within the Pharmacy Notes section, alongside the prescription authored by the healthcare professional.

Following prescription entry, the eRx interface—within either the EHR or POC system—transmits a distribution signal to the electronic discount request platform, specifying the selected delivery channel for the co-pay message. Upon receipt of the electronic prescription and associated discount information, the pharmacy processes the transaction by applying the co-pay discount to the patient's bill prior to dispensing the medication. To ensure proper application of the discount, all affordability message content, including electronic discount and benefit details, must be embedded within the pharmacy notes section. It is the responsibility of the publisher to implement this integration step during system setup, ensuring the discount information is properly inserted into the pharmacy notes in accordance with the platform's technical specifications.

For distribution of electronic discounts to a patient portal, the e-prescription (eRx) page within the EHR system or Point-of-Care (POC) platform—integrated with the electronic discount request platform—must be configured to transmit the co-pay discount and associated benefit information directly to the inbox section of the patient portal. This transmission occurs in conjunction with the HCP's prescription script, allowing the patient to access the electronic discount alongside their prescribed medication details within the portal interface.

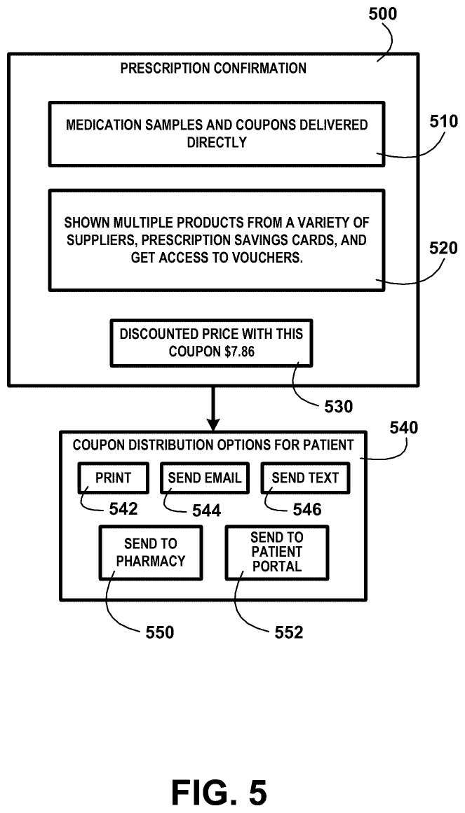

FIG. 5 illustrates a block diagram of one embodiment of a prescription confirmation interface 500. In this embodiment, the system may present the healthcare professional (HCP) 110 with various supplemental options, including medication samples and electronic discounts delivered directly through the system 510. The HCP may be shown multiple available products from different suppliers, along with prescription savings cards and access to co-pay vouchers 520. In the illustrated example, the prescription confirmation interface displays the reduced out-of-pocket cost—such as a discounted price of $7.86—based on the applicable electronic discount 530. The system also presents multiple distribution options 540 for delivering the discount to the patient, including print 542, email 544, text message 546, transmission to the pharmacy 550, and delivery via the patient portal 552. Upon selection of a distribution method, the eRx system (EHR or POC) sends a distribution signal to the electronic discount request platform, indicating the selected delivery channel. The patient then receives and displays the electronic prescription with the applied co-pay discount, which the pharmacy uses to apply the discount to the bill before dispensing the prescribed medication.

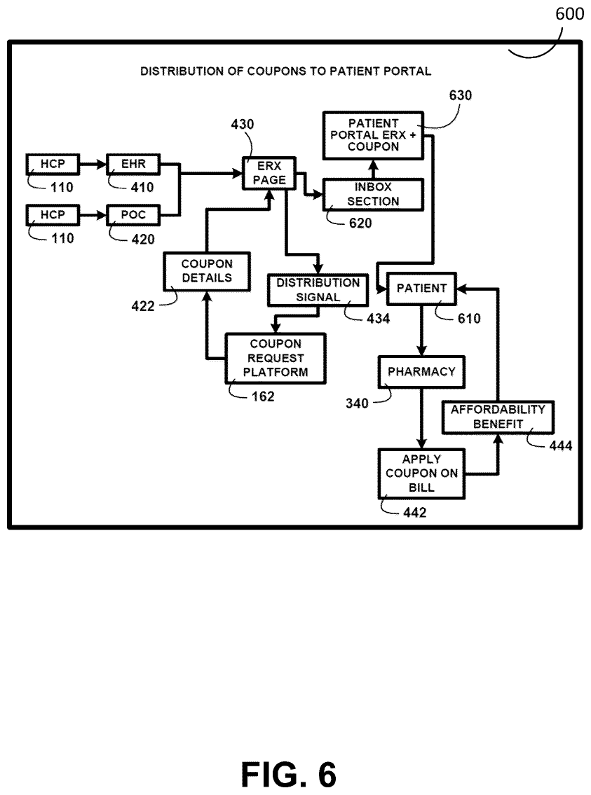

FIG. 6 illustrates a block diagram representing one embodiment of the distribution of electronic discounts to a patient portal 600. In this embodiment, the healthcare professional (HCP) 110 accesses the electronic prescription (eRx) page 430 via either the Electronic Health Record (EHR) system 410 or the Point-of-Care (POC) system 420 and enters a prescription. Upon submission, the eRx page transmits a distribution signal 434 to the electronic discount request platform 162. The platform processes the request and returns the applicable electronic discount details 422. These discount details are then routed to the inbox section 620 of the patient portal 630, where they are displayed in conjunction with the electronic prescription. The patient 610 can subsequently forward the discount information to their designated pharmacy 340. The pharmacy receives the prescription and discount data, applies the discount to the transaction 442, and communicates the affordability benefit 444 to the patient 610 prior to dispensing the medication.

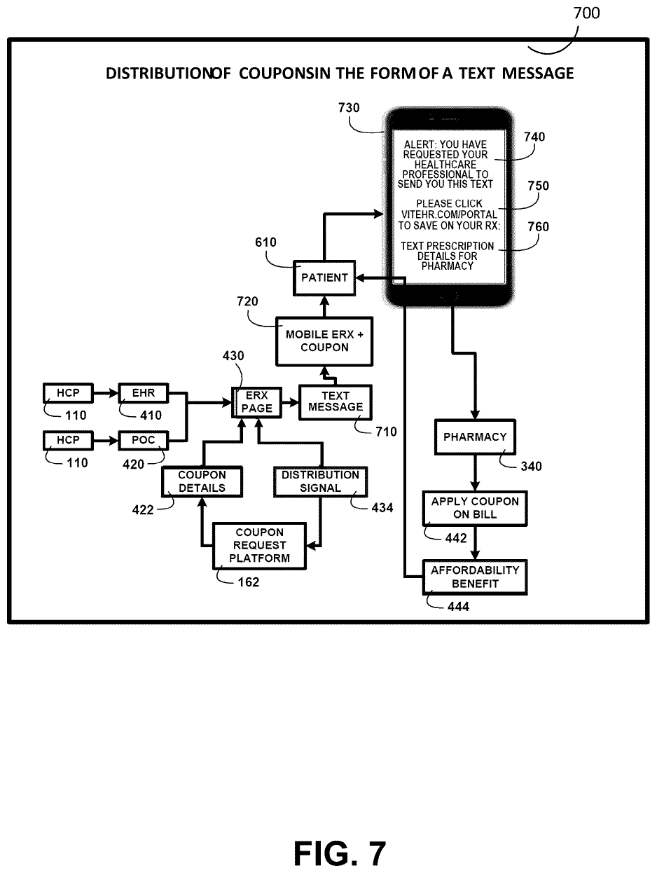

FIG. 7 presents a block diagram illustrating one embodiment of electronic discount distribution via text message 700. In this embodiment, the healthcare professional (HCP) 110 accesses the electronic prescription (eRx) page 430 through either the Electronic Health Record (EHR) system 410 or the Point-of-Care (POC) system 420 and enters a prescription. Upon prescription entry, the eRx page transmits a distribution signal 434 to the electronic discount request platform 162. The platform processes the request and returns the applicable electronic discount details 422. These details are then formatted into a text message 710 containing both the prescription and discount information 720, which is delivered to the patient 610 on their mobile device 730. Upon opening the message, the patient is presented with a confirmation alert 740 stating: “You have requested your healthcare professional to send you this text.”

The text message 710 includes a link, such as “Please click vitEHR.com/portal to save on your Rx” 750, directing the patient to access their co-pay discount. The message also contains relevant prescription details intended for use by the pharmacy 760. Upon receiving the text, the patient may present the message in person or forward it electronically to the designated pharmacy 340. The text message includes embedded instructions for the pharmacy 340 to apply the associated electronic discount to the patient's bill 442 and to deliver the corresponding affordability benefit 444, in accordance with one embodiment of the system.

For distribution of electronic discounts via text message, the electronic prescription (eRx) page within the EHR or POC system—integrated with the electronic discount request platform—facilitates transmission of the discount in text format directly to the patient's mobile device, along with the associated prescription information from the healthcare professional (HCP). Once the prescription is entered, the eRx system generates a distribution signal specifying the selected channel (i.e., text message) and sends it to the electronic discount request platform. The patient then receives and displays the e-prescription containing the applied co-pay message (electronic discount). Upon presentation at the pharmacy, the pharmacy applies the discount to the patient's bill before dispensing the prescribed medication.

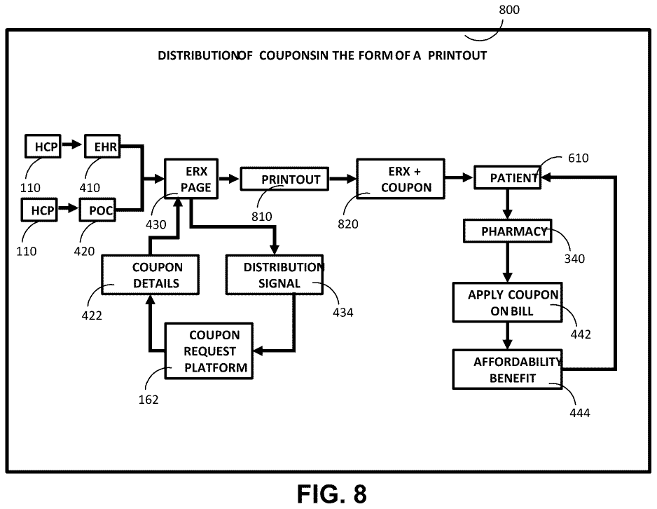

FIG. 8 illustrates a block diagram representing one embodiment of electronic discount distribution in the form of a printed document 800. In this embodiment, the healthcare professional (HCP) 110 accesses the electronic prescription (eRx) page 430 via either the EHR system 410 or the Point-of-Care (POC) system 420, as previously shown in FIG. 4A. Upon entering the prescription, the eRx page generates and transmits a distribution signal 434 to the electronic discount request platform 162. The request platform processes the prescription information and returns the corresponding electronic discount details 422. These details are prepared for inclusion in a printout for delivery to the patient.

The electronic discount details 422, as shown in FIG. 4A, are formatted for print and delivered to the pharmacy 810 in the form of a combined document containing both the electronic prescription (eRx) and the associated co-pay discount. This printed document includes the relevant discount information and affordability benefit details 820, and is generated at the HCP's location or medical facility for manual handoff to the patient 610, as shown in FIG. 6. The patient then presents this printout at the designated pharmacy 340, as depicted in FIG. 3. Upon receipt, the pharmacy applies the co-pay discount to the transaction, adjusting the bill 442 accordingly and delivering the affordability benefit 444 to the patient, as described in FIG. 4A and in accordance with one embodiment of the system.

For distribution of electronic discounts in printed form, the electronic prescription (eRx) page—within the EHR or POC system integrated with the electronic discount request platform—may be configured to generate a printout of the applicable discount at the healthcare professional's (HCP's) location or medical facility. This printed document, which includes the co-pay discount along with the HCP's prescription script, is manually delivered to the patient. Upon prescription entry, the eRx system transmits a distribution signal specifying the print delivery channel to the electronic discount request platform. The patient then presents the printed eRx containing the applied co-pay message (electronic discount) at the pharmacy, where the discount is applied to the transaction before the medication is dispensed.

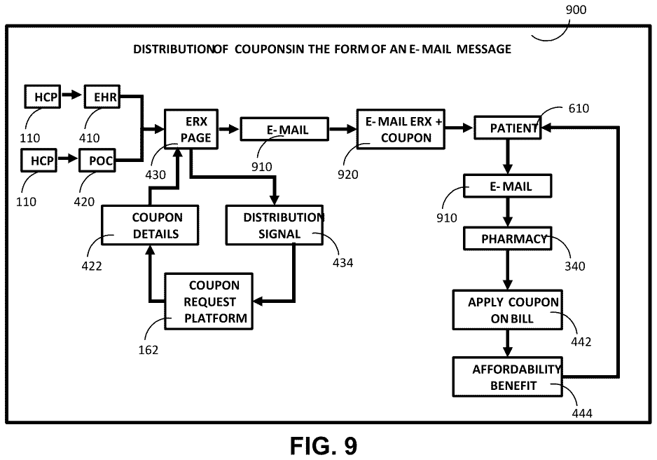

FIG. 9 illustrates a block diagram of one embodiment for distributing electronic discounts via email. In this embodiment, the healthcare professional (HCP) 110 accesses the electronic prescription (eRx) page 430 through either the Electronic Health Record (EHR) system 410 or the Point-of-Care (POC) system 420 and enters a prescription. Upon submission, the eRx page sends a distribution signal 434 to the electronic discount request platform 162. The platform processes the request and returns the applicable electronic discount details 422. These details are then compiled with the eRx information and sent to the patient 610 via email 910, forming a combined message 920. The patient receives the email and may forward it to their designated pharmacy 340. Upon receipt, the pharmacy applies the electronic discount to the patient's bill 442 and communicates the affordability benefit 444 to the patient, in accordance with the described embodiment. For distribution of electronic discounts via email, the electronic prescription (eRx) page within the EHR or POC system—integrated with the electronic discount request platform—may be configured to transmit the applicable co-pay discount to the patient's email client. This email includes both the electronic discount and the healthcare professional's (HCP's) prescription script, enabling the patient to access and forward the discount information to a pharmacy for application at the point of sale.

Upon selection of email as the distribution method, the e-prescription (eRx) interface within the EHR or POC system transmits a distribution signal—indicating the chosen channel type—to the electronic discount request platform. The platform then delivers the electronic discount along with the eRx to the patient via email. The patient can present this email, containing the co-pay message and prescription details, at the pharmacy. The pharmacy applies the electronic discount to the patient's bill before dispensing the prescribed medication. After the co-pay message has been successfully distributed, the system collects and aggregates relevant program metrics. This data is recorded on the Publisher Dashboard via the electronic discount request platform, and the associated EHR or POC system also tracks discount utilization for reporting and analytics purposes.

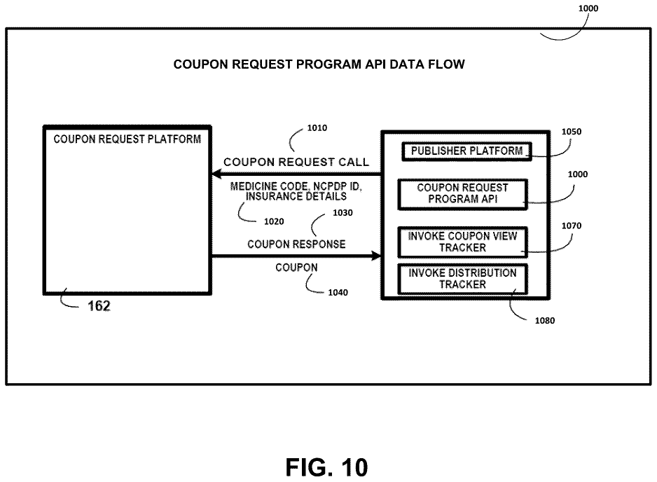

FIG. 10 illustrates a block diagram of one embodiment of an electronic discount request call workflow. In this embodiment, the electronic discount request platform 162 receives a request call 1010 that includes critical information such as the medication code, NCPDP ID, and insurance details 1020. The platform processes this information and returns an electronic discount response 1030 containing the applicable electronic discount 1040. This request-response interaction is managed via an electronic discount request program API data flow 1000. The API 1060, which is integrated into the publisher platform 1050, enables real-time communication with the electronic discount request platform 162. Upon receiving the discount response, the publisher platform 1050 invokes two tracking functions: the electronic discount view tracker 1070 and the distribution tracker 1080, which respectively track healthcare professional (HCP) engagement with the discount and the subsequent distribution activity, in accordance with one embodiment.

The data flow between the electronic discount request platform 162 and the associated publisher platform is central to the co-pay affordability message distribution process. Initially, the publisher partner integrates the electronic discount request platform's Application Programming Interface (API) into its system—whether an EHR system or a Point-of-Care (POC) platform—and completes all necessary platform and ad slot configuration steps. Once integration is complete, the API enables a seamless workflow comprising the following stages: (1) the publisher platform initiates an electronic discount request call to the electronic discount request platform; (2) the platform responds with the applicable electronic discount data; (3) the publisher platform invokes the electronic discount view tracker by accessing the electronic discount view URL; and (4) the publisher platform invokes the distribution tracker by accessing the distribution URL. This data flow ensures proper tracking of both discount visibility and delivery actions across the integrated platforms.

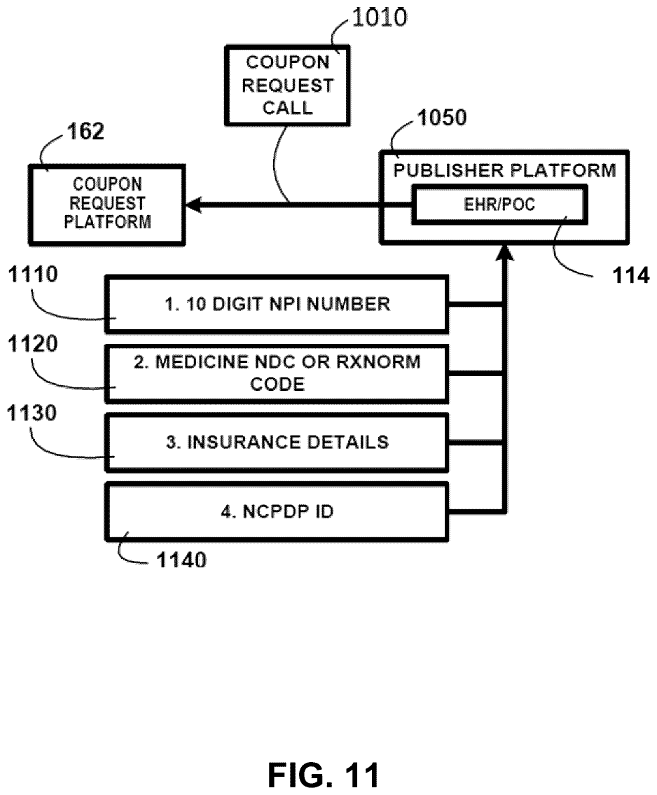

FIG. 11 illustrates a block diagram providing an overview of the publisher platform in one embodiment. In this configuration, the publisher platform 1050 is triggered by an eRx event initiated through the EHR or POC system 114. Upon such an event, the publisher platform generates and transmits an electronic discount request call 1010 to the electronic discount request platform 162. This request call includes the following key data elements: (1) the healthcare professional's 10-digit National Provider Identifier (NPI) 1110, (2) the medication identifier, which may be either a National Drug Code (NDC) or RxNorm code 1120, (3) the patient's insurance information 1130, and (4) the National Council for Prescription Drug Programs (NCPDP) pharmacy identifier 1140. Prior to this workflow, the electronic discount request platform API must be fully integrated into the publisher platform 1050 to enable automatic generation and transmission of the discount request call 1010 whenever a healthcare professional initiates an electronic prescription event within the EHR or POC system.

The electronic discount request call 1010, which is triggered from the EHR system or POC platform, is transmitted to the electronic discount request platform 162 and typically includes the following core data elements: (1) the National Provider Identifier (NPI) corresponding to the prescribing healthcare professional (HCP); (2) the medication identifier, which may be a National Drug Code (NDC) or RxNorm code, for the drug prescribed in the electronic prescription (eRx); (3) the patient's insurance information, including whether coverage exists, the type of insurance, and the name of the insurance provider; and (4) the National Council for Prescription Drug Programs (NCPDP) identifier associated with the selected pharmacy. These data points are necessary for accurately matching and returning an applicable co-pay electronic discount.

The National Provider Identifier (NPI) of the healthcare professional (HCP) is a 10-digit numeric string that uniquely identifies the HCP accessing the EHR or POC interface where the electronic discount will be rendered. To ensure privacy and comply with data protection protocols, the NPI is anonymized by applying a SHA-256 cryptographic hash function. This process generates a 64-character hexadecimal digest from the original NPI input, effectively obscuring the provider's identity while preserving the ability to track and match transactions within the system. Hashing the NPI is a required business rule implemented as part of the discount platform's data handling workflow.

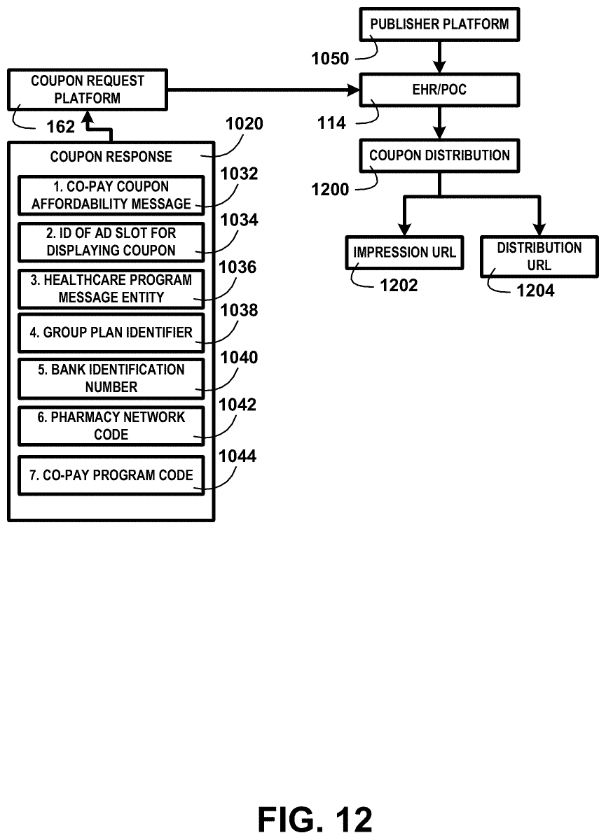

The ad server component associated with the co-pay message distribution and electronic discount request platform is used for processing incoming discount request calls. Upon receiving a request, the ad server evaluates the submitted data and returns an appropriate response to the originating publisher platform. Specifically, the electronic discount response—transmitted from the electronic discount request platform 162 to the publisher platform 1050—includes a co-pay electronic discount if one is available and applicable for the patient's prescription. FIG. 12 illustrates a block diagram representing this process in one embodiment, where the electronic discount request platform 162 sends an electronic discount response 1030 to the publisher platform 1050.

The publisher platform 1050—which may be an EHR or POC system 114—receives the electronic discount response from the electronic discount request platform. This response includes multiple data fields necessary for rendering and tracking the co-pay message. Specifically, the response contains: (1) the co-pay electronic discount affordability message 1032, (2) the identifier of the advertisement slot where the electronic discount will be displayed 1034, (3) the healthcare program message entity 1036, (4) the group plan identifier 1038, (5) the bank identification number (BIN) 1040, (6) the pharmacy network code 1042, and (7) the co-pay program code 1044. The response also includes metadata supporting distribution, such as the electronic discount distribution ID 1200, an impression tracking URL 1202, and a distribution tracking URL 1204, all of which enable accurate tracking and reporting of co-pay message visibility and delivery.

Additionally, the electronic discount response includes supplementary ad-serving metadata required for proper rendering and delivery of the co-pay message within the publisher platform, which may be an EHR system or POC platform. These details include: the identifier of the advertisement slot designated for displaying the co-pay electronic discount; the unique identifier for the healthcare program member entity associated with the discount; the group or plan identifier within the broader affordability messaging program; the bank identification number (BIN) used for insurance or pharmacy benefit processing; the pharmacy network code indicating where the discount is valid; and the internal co-pay program ID used by the electronic discount request platform. This information supports precise targeting, compliance, and fulfillment within the overall system workflow.

Upon receiving the electronic discount response from the electronic discount request platform 162, the publisher platform 1050—such as the EHR or POC system 114—invokes two specific tracking URLs: (1) the Impression URL and (2) the Distribution URL. The Impression URL is used to track whether the co-pay electronic discount advertisement has been displayed within the publisher interface and viewed by the healthcare professional (HCP). This tracking is essential for recording ad impressions and is typically used for billing and performance analytics. In contrast, the Distribution URL is triggered when the HCP interacts with the co-pay message, such as by clicking to transmit the discount to a patient. This interaction is used for conversion tracking, allowing the system to monitor actual distribution events and assess user engagement with the co-pay messaging functionality.

The co-pay affordability message distribution process concludes when the healthcare professional (HCP) successfully delivers the electronic discount to the patient. The method of distribution—whether via Pharmacy Notes, Patient Portal, Text Message, Email, or Print—is predetermined by the publisher partner and configured within the publisher's EHR or POC platform during the integration phase. Once the electronic discount has been distributed through the designated channel, the patient may present it at a participating pharmacy of their choice to redeem the applicable financial benefit as defined by the terms of the co-pay electronic discount.

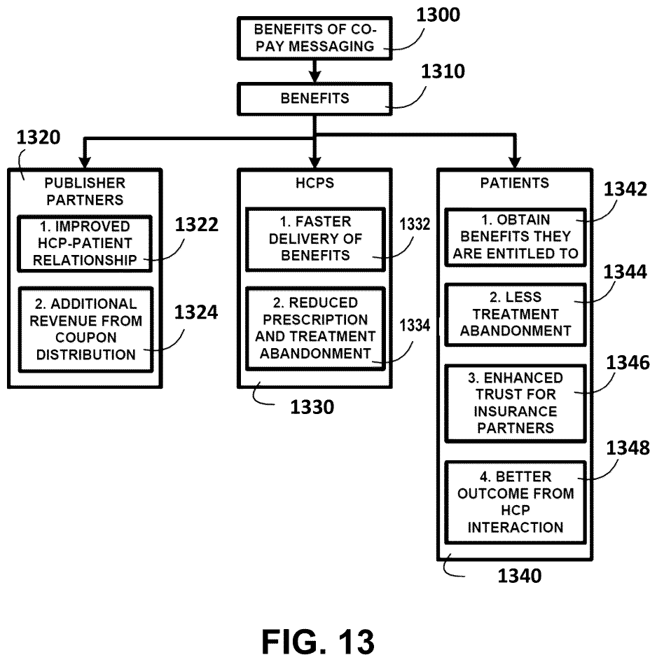

FIG. 13 illustrates a block diagram outlining the benefits of co-pay messaging according to one embodiment. The benefits 1300 span across all key stakeholders involved in the system. Publisher partners 1320 realize advantages such as (1) improved relationships between healthcare professionals (HCPs) and patients 1322, and (2) increased revenue opportunities resulting from electronic discount distribution 1324. For HCPs 1330, the system enables (1) faster and more efficient delivery of affordability benefits to patients 1332, and (2) a measurable reduction in prescription abandonment and discontinuation of treatment 1334. Patients 1340 benefit from (1) access to co-pay savings to which they are entitled 1342, (2) decreased likelihood of abandoning treatment due to cost 1344, (3) increased trust in insurers and care providers 1346, and (4) improved overall outcomes from clinical interactions with HCPs 1348.

The co-pay message distribution system delivers significant benefits to all participating stakeholders, including healthcare professionals (HCPs), patients, and publisher partners. For publisher partners—such as EHR systems and Point-of-Care (POC) platforms—the system enhances the quality of HCP-patient interactions, leading to increased provider satisfaction and engagement. Additionally, publisher partners can capitalize on revenue-generating opportunities through a revenue-sharing model tied to the successful distribution and utilization of electronic discounts within their integrated platforms. This model incentivizes active participation in delivering affordability messaging while contributing to improved health outcomes and system-wide efficiencies.

Healthcare professionals (HCPs) benefit from real-time access to a wide range of cost-saving, benefit, and affordability programs through the co-pay message distribution system. This streamlined access reduces administrative overhead typically associated with locating and delivering patient financial assistance, enabling HCPs to more efficiently distribute applicable co-pay discounts during clinical workflows. As a result, HCPs can deliver financial benefits to patients more quickly, improving the overall care experience. Additionally, increased electronic discount distribution—particularly during the first year of system adoption—contributes to enhanced patient affordability and a measurable reduction in prescription abandonment and treatment non-adherence.

Patients benefit directly from the co-pay message distribution system by gaining access to affordability programs that align with their selected insurance plans. When patients are able to utilize co-pay discounts effectively, they are more likely to continue their prescribed treatment regimens, thereby reducing instances of therapy abandonment due to cost concerns. Additionally, consistent access to co-pay benefits fosters increased trust between patients and their healthcare and insurance providers. The enhanced and timely distribution of electronic discounts contributes to improved patient-HCP interactions and ultimately supports better clinical outcomes.

The electronic discount request call initiated by the publisher platform includes key data elements necessary for processing the discount. Specifically, it transmits the National Provider Identifier (NPI) of the healthcare professional (HCP), the medication code (such as NDC or RxNorm) corresponding to the drug prescribed during the patient's caregiving session, and the patient's insurance details. Depending on the configuration specified by the publisher, the request call may also include the National Council for Prescription Drug Programs (NCPDP) ID to identify the dispensing pharmacy. These data elements enable the electronic discount request platform to match and return an appropriate co-pay offer.

The foregoing has described the principles, embodiments, and modes of operation of the present invention. However, the invention should not be construed as being limited to the embodiments discussed. The above-described embodiments should be regarded as illustrative rather than restrictive, and it should be appreciated that variations may be made in those embodiments by workers skilled in the art without departing from the scope of the present invention as defined by the following claims.

What is claimed is:

1. A co-pay discount delivery system, comprising:
a non-transitory memory for storing instructions; and
a processor operatively coupled to the memory and configured to execute the instructions to:
detect, through an electronic prescription interface within an electronic point-of-care (POC) platform, a prescription entry comprising a medication identifier;
generate an electronic discount request including the medication identifier, a hashed healthcare provider identifier, and patient insurance data;
transmit the electronic discount request over a network to a remote discount server;
receive, from the remote discount server, discount response data comprising at least one of a branded or a generic co-pay discount associated with the medication identifier;
associate the received discount response data with the prescription;
select at least one delivery channel from the group consisting of: a pharmacy note field, a patient portal message, a short message service (SMS) message, an email, and a print command;
transmit the associated discount and prescription data through the selected delivery channel; and
record a log entry comprising redemption or usage data and transmit the log entry to a reporting interface.

2. The co-pay discount delivery system of claim 1, wherein the processor is further configured to determine whether a branded co-pay discount is available for the medication identifier and, if not available, to select a generic co-pay discount.

3. The co-pay discount delivery system of claim 1, wherein the prescription entry includes a National Drug Code (NDC) or RxNorm code as the medication identifier.

4. The co-pay discount delivery system of claim 1, wherein the hashed healthcare provider identifier is generated using a secure hashing algorithm applied to a National Provider Identifier (NPI).

5. The co-pay discount delivery system of claim 1, wherein the reporting interface comprises a publisher dashboard configured to display redemption activity in real time.

6. The co-pay discount delivery system of claim 1, further comprising discount content with instructions to be applied by a pharmacy system to reduce patient out-of-pocket costs prior to medication dispensation.

7. A co-pay discount delivery system, comprising:
a prescription detection processor configured to interface with an electronic prescription page within an electronic point-of-care (POC) platform, the prescription detection processor being configured to detect a prescription entry comprising a medication identifier selected from the group consisting of a National Drug Code (NDC) and an RxNorm code;
a request generation processor operatively coupled to the prescription detection processor, the request generation processor configured to generate an electronic discount request comprising the medication identifier, a hashed healthcare provider identifier, and patient insurance data;
a network communication processor configured to transmit the electronic discount request to a remote discount platform and receive, in response, discount response data comprising at least one of a branded or generic co-pay discount associated with the medication identifier associated with a prescribed medication;
a delivery routing processor configured to associate the discount response data with the prescription and to deliver the discount response data via at least one of a plurality of delivery channels, the delivery channels comprising: a pharmacy note field, a patient portal, a text message, an email message, and a printed document;
a redemption tracking processor configured to monitor usage of the delivered co-pay discount and to collect and format redemption activity data; and
a reporting processor configured to transmit the redemption activity data to a remote publisher dashboard for analysis and display.

8. The co-pay discount delivery of claim 7, wherein the medication identifier is a National Drug Code (NDC), and the prescription detection processor is further configured to extract the NDC automatically upon medication selection from a prescribing interface.

9. The co-pay discount delivery of claim 7, wherein the hashed healthcare provider identifier is generated using a cryptographic hash function applied to the healthcare provider's National Provider Identifier (NPI).

10. The co-pay discount delivery of claim 7, wherein the request generation processor is further configured to include in the electronic discount request an insurance provider name and an insurance plan type.

11. The co-pay discount delivery of claim 7, wherein the network communication processor is configured to determine whether a branded co-pay discount is available for the prescribed medication and, if not, to retrieve a generic co-pay discount as a fallback.

12. The co-pay discount delivery of claim 7, wherein the delivery routing processor is configured to insert the discount response data into a pharmacy note field of the electronic prescription for delivery to the selected pharmacy.

13. The co-pay discount delivery of claim 7, wherein the delivery routing processor is further configured to transmit a text message containing the co-pay discount and prescription information to a mobile device associated with the patient.

14. The co-pay discount delivery of claim 7, wherein the delivery routing processor is configured to transmit the co-pay discount to the patient portal of the EHR or POC platform, including an embedded link to retrieve the discount at a participating pharmacy.

15. The co-pay discount delivery of claim 7, wherein the redemption tracking processor is configured to collect redemption status, timestamp, pharmacy identifier, and the distribution channel used.

16. The co-pay discount delivery of claim 7, wherein the reporting processor is further configured to generate real-time visualizations of redemption activity for display on a publisher-facing dashboard.

17. A co-pay discount delivery system, comprising:
a prescription entry processor configured to detect a prescription entry made by a healthcare professional into an electronic prescription (eRx) interface of an electronic health record (EHR) point-of-care (POC) platform, the prescription including a National Drug Code (NDC);
a discount request processor operatively coupled to the prescription entry processor, the discount request processor configured to generate an electronic discount request including the NDC, a hashed National Provider Identifier (NPI) of the healthcare professional, and insurance plan data for the patient, and to transmit the request to a remote discount platform over a network;
a discount retrieval processor configured to receive, from the discount platform, co-pay discount response data comprising either a branded or generic electronic discount applicable to the NDC;
a distribution processor configured to insert the received co-pay discount into a pharmacy instruction field of the eRx and transmit the eRx and discount to a pharmacy system electronically; and
a tracking processor configured to log redemption of the co-pay discount at the pharmacy and transmit redemption data to a remote publisher dashboard.

18. The co-pay discount delivery system of claim 17, wherein the discount request processor is further configured to determine whether a branded co-pay discount is available for the NDC and to select a generic co-pay discount only if a branded discount is not available.

19. The co-pay discount delivery system of claim 17, wherein the distribution processor is further configured to transmit the co-pay discount to no more than two of the following distribution channels: a patient portal inbox and a mobile device via text message.

20. The co-pay discount delivery system of claim 17, wherein the tracking processor is further configured to transmit, to the publisher dashboard, data comprising at least: a timestamp of redemption, a pharmacy identifier, and the type of co-pay discount applied.
